(19) United States
(12) Patent Application Publication
(10) Pub. No.: US 2011/0263450 A1
(43) Pub. Date: Oct. 27, 2011

(54) ALZHEIMER'S DISEASE BIOMARKERS

(75) Inventors: Kevin Jeffrey Barnham, Parkville (AU); Victor Luis Villemagne, Parkville (AU); Kayla Azorena Perez Camacaro, Parkville (AU)

(73) Assignee: THE UNIVERSITY OF MELBOURNE, Parkville, Victoria (AU)

(21) Appl. No.: 13/121,119

(22) PCT Filed: Sep. 25, 2009
(86) PCT No.: PCT/AU09/01279

§ 371 Date: Jul. 15, 2011

(30) Foreign Application Priority Data
Sep. 26, 2008 (AU) 2008905035

(51) Int. Cl. C40B 30/04; C07K 2/00; C07K 14/435; C40B 40/10; G01N 33/53

(52) U.S. Cl. 506/9; 506/18; 436/501; 530/350; 530/300

(57) ABSTRACT

The invention provides a biomarker for qualifying Alzheimer's disease status, the biomarker being detectable in a biological sample containing blood cellular elements and being derived from amyloid precursor protein or amyloid β peptide. Particularly the biomarker includes an Aβ 1-42 or Aβ 1-43 species (monomer) and, or an Aβ 1-42 dimer. An alternative or additional biomarker includes an APP cathepsin D cleavage product or includes a biomarker identifiable by a peak of molecular weight of about 9962 or 9980 Daltons when identified by SELDI-TOF MS utilising antibody WO2.

### FIELD

The present invention is in the field of diagnostics. More specifically the invention relates to Alzheimer's disease (AD) biomarkers and methods for determining AD status using AD biomarkers.

### BACKGROUND

Alzheimer's disease (AD) is the most common age related dementia. Definitive diagnosis relies on demonstrating the presence of amyloid-β (Aβ) plaques and tau neurofibrillary tangles at autopsy due to a lack of valid biomarkers for AD. Diagnosis of probable AD is currently based on progressive memory impairment and decline in at least one other cognitive domain, requiring careful observation of behavioural changes and neuropsychological testing. Moreover, clinical symptoms are usually evident only after substantial and probable irreversible synaptic and neuronal loss has occurred making the success of potential disease-modifying therapeutic approaches difficult. The ability to detect preclinical or early stage disease through reliable biomarkers for AD would allow treatment and management of the disease to begin earlier.

The lack of valid biomarkers for AD also hampers the development of effective therapies for AD, as misdiagnosed patients are often enrolled into clinical trials. Additionally, the assessment of outcomes of these trials is difficult to define and often relies on the more subjective neuropsychometric tests. To overcome the variability that is inherent in these tests, large sample sizes are essential, additionally long timeframes are usually required to see subtle changes in subject performance in the neuropsychometric tests; all of this dramatically increases the cost of the trials. The ability to detect preclinical or early stage disease through reliable biomarkers for AD would allow for more efficient clinical trials to be designed and monitored.

The best biomarkers for AD to date are based on ELISA measurements of tau and Aβ₄₂ in cerebrospinal fluid (CSF) and of Aβ burden in the brain with ¹¹C-PiB PET. Nonetheless, they are associated with serious drawbacks such as poor sensitivity or specificity, high costs associated with either the tests themselves or as a consequence of a lack of one simple test providing an accurate diagnosis for AD; or relying on invasive procedures.

With disease modifying therapies for AD undergoing clinical trials, there is a social, economic and even a moral imperative to identify biomarkers that can detect features of the disease in at-risk individuals in the earliest possible stage, so anti-AD therapy can be administered at a time when the disease burden is mild and it may prevent or delay functional and irreversible cognitive loss.

Ciphergen Biosystems, Inc. has several patent applications directed to biomarkers for AD, for example the applications published as WO 2006/113289, WO 2006/138325 and WO2005/047484. All of these applications describe using SELDI mass spectrometry to detect protein biomarkers for AD in cerebrospinal fluid (CSF) and serum. However tests looking for AD biomarkers in plasma are contradictory at best.

There is a need, therefore, to identify AD biomarkers that are sensitive and specific to AD and provide for AD diagnosis in a rapid and non-invasive manner.

### SUMMARY

The present invention provides polypeptide biomarkers that are differentially present in subjects having AD versus subjects free of the disease. In addition, the present invention provides methods of using the biomarkers to qualify AD status in a subject. The present invention also provides methods for identifying AD therapeutics and monitoring progression of AD.

The first aspect provides a biomarker for qualifying Alzheimer's disease status, said biomarker being detectable in a biological sample containing blood cellular elements and being derived from amyloid precursor protein or amyloid β peptide.

The second aspect provides a biomarker for qualifying Alzheimer's disease status, said biomarker being detectable in a biological sample containing blood cellular elements and being selected from the list of biomarkers presented in Table 1.

TABLE 1
The molecular weight of peaks identified utilizing the antibodies
WO2 and 4G8 in the SELDI-TOF MS where the intensities were
statistically different between AD and HC subjects.

| | |
|---|---|
| WO2 | 4G8 |

| Peak | | Peak | |
|---|---|---|---|
| molecular | | molecular | |
| weight | p value | weight | p value |
| 4529/4535 | 0.07/0.0008 | | |
| 4625/4631 | 0.003/0.0107 | | |
| 5289/5297 | 0.028/0.0439 | | |
| 9058/9070 | <0.0001/0.0005 | 9058/9070 | 0.001/0.0011 |
| 9962/9980 | 0.002/0.0021 | | |
| 10255/10293 | 0.0037/0.004 | 10254/10292 | 0.022/0.034 |
| 11310/11330 | 0.001/0.0014 | 11310/11330 | 0.044/0.0435 |
| 11346/11364 | 0.001/0.0055 | | |
| 11432/11453 | 0.014/0.0163 | | |
| 12310/12330 | 0.002/0.0082 | 12310/12330 | 0.024/0.0241 |
| 12768/12787 | 0.003/0.0028 | | |
| 12834/12859 | 0.008/0.0114 | | |
| 15315/15339 | 0.07/0.0135 | | |
| 15524/15546 | 0.06/0.0057 | | |

The third aspect provides a method for qualifying Alzheimer's disease status in a subject, the method comprising assaying a biological sample from the subject, the biological sample comprising blood cellular elements, for a biomarker according to the first or second aspect and correlating the result of the assay with Alzheimer's disease status.

The fourth aspect provides use of a biomarker according to the first or second aspect for qualifying Alzheimer's disease status in a subject by assaying a biological sample from the subject for said biomarker, said biological sample comprising blood cellular elements.

Whereas detection of one biomarker is in most cases sufficient to reliably diagnose AD, detection of two or more biomarkers can increase the sensitivity and robustness of the method. Accordingly in an embodiment of the third and fourth aspects, the method or use comprises assaying a plurality of biomarkers.

In an embodiment of the third and fourth aspects, the method or use comprises assaying a plurality of biomarkers, a first biomarker being a peptide involved in an amyloidogenic pathway and a second biomarker being a peptide involved in a non-amyloidogenic pathway and wherein an increase in the first biomarker and a decrease in the second biomarker compared to control is indicative of Alzheimer's disease status.

If all biomarkers present in a sample are measured a pattern of biomarkers will result and this pattern may be compared with patterns previously calibrated with AD status. This allows rapid detection of AD status, either using appropriate software for statistical analysis including pattern recognition or in many cases detection of AD status by simple visual inspection.

In an embodiment of the first aspect the biomarker comprises an Aβ 1-42 or Aβ 1-43 species (monomer) and, or an Aβ -1-42 dimer, wherein an increase in the monomer or dimer compared to control is indicative of AD.

In another embodiment of the first aspect the biomarker has a peak of molecular weight of about 9962 or 9980 Daltons when identified by SELDI-TOF MS utilising antibody WO2 or 4G8, wherein a decrease in the 9962 or 9980 biomarker is predictive of AD status. This molecular weight appears to correspond to an APP fragment resulting from cathepsin D activity. It has previously been shown that the activity of this protease is decreased in the blood of AD subjects.

In an embodiment of the third or fourth aspect the method or use comprises assaying for an Aβ monomer and or Aβ dimer, wherein an increase in the monomer or dimer compared to control is predictive of AD.

In another embodiment of the third or fourth aspect the method or use comprises assaying for the presence of biomarker derived from APP or Aβ and having a peak of molecular weight of about 9962 or 9980 Daltons when identified by SELDI-TOF MS utilising antibody WO2 or 4G8, wherein a decrease in the 9962 or 9980 biomarker is predictive of AD status.

In an embodiment of the third or fourth aspect the method or use comprises assaying for a APP cathepsin D cleavage product, wherein a decrease in the cleavage product is predictive of AD.

In a preferred embodiment of the third or fourth aspect the method or use comprises assaying for the presence of an Aβ monomer and or Aβ dimer together with assaying for the 9962 or 9980 peak or cathepsin D cleavage product, wherein an increase in the Aβ monomer or dimer and a decrease in the 9962 or 9980 peak or cathepsin D cleavage product compared to control is indicative of Aβ status.

The inventors were investigating biomarkers for AD. Due to the important role that APP and Aβ have in the disease the inventors considered that APP or Aβ species should provide suitable biomarkers in biological samples.

However their studies determined that plasma samples do not provide APP/Aβ biomarker profiles that are substantially different between AD and non-AD (control) samples. Rather than consider that APP and Aβ were not suitable biomarkers the inventors went on to study different samples. The inventors consider that APP/Aβ is membrane bound, as are the proteases that generate Aβ and a range of studies have shown that many of the deleterious biological effects attributable to Aβ are due to the interaction of oligomeric neurotoxic Aβ peptides with cell membranes.

Accordingly the inventors used the usually discarded membrane rich fraction for detecting APP/Aβ biomarkers and found that a biological sample useful for detecting APP/Aβ biomarkers and therefore allowing qualification of AD status is any sample that contains cellular material, particularly whole blood, white blood cells, red blood cells, platelets or exosomes or a combination thereof.

A sample containing blood cells and platelets was analysed by SELDI-TOF MS using antibody capture using commercially available antibodies WO2 epitope Aβ residues 4-8 and 4G8 epitope Aβ residues 17-21. The spectra obtained appeared to be different between AD and control but the spectra were hard to read due to the presence of background interference. The test was further refined by treating the biological sample with urea, buffer and a non-ionic detergent to break up any potential protein/protein or protein/membrane interactions. If the cellular sample was whole blood preferably the plasma was separated from the cellular elements prior to extraction. A preferred extraction process is described in Example 1. Samples treated in this way gave higher resolution spectra and showed detectable biomarkers based on APP and Aβ which can be differentiated in AD and control samples. Another option would have been to further fractionate the blood sample into cell types, as it is probable that the biomarkers are more prevalent on a particular cell type. Initial studies using samples of white blood cells showed improved signal to noise in the spectra that would be predictive of improved sensitivity and hence specificity. Accordingly the urea/detergent treatment of the sample need not be necessary if particular cell fractions are used or if particularly sensitive antibodies to the biomarkers are available.

The AD status is preferably selected from AD, non-dementia, non-AD dementia and MCI. Non-AD dementia included Lewy body dementia (LBD) and frontotemporal dementia (FTD).

The invention may further comprise managing subject treatment based on AD status determined according to the method of the third aspect and this may optionally comprise further qualifying AD status after treatment to determine efficacy of treatment.

The fifth aspect provides a kit comprising a solid support comprising at least one capture agent attached thereto, wherein the capture reagent binds at least one biomarker according to the first or second aspect and instructions for using the solid support to detect the at least one biomarker.

The solid support comprising a capture agent may be a SELDI chip.

Preferably the solid support comprises a Aβ/APP specific antibody, for example WO2 or 4G8 coupled to a SELDI antibody chip, for example PS10.

The sixth aspect provides a method of identifying biomarkers for amyloid diseases involving Aβ accumulation, the method comprising; performing an APP/Aβ capture assay on cellular samples from subjects diagnosed as diseased or control using a Aβ/APP specific antibody or other specific capture agent, assaying bound proteins using mass spectrometry, comparing the peaks generated from control samples to those from disease samples and determining peaks that are present or the intensity of the peak significant altered in one sample but not the other.

The seventh aspect provides a purified biomolecule selected from the biomarkers listed in Table 1 as identified by the method of the sixth aspect.

Persons skilled in the art will appreciate that different antibodies will give rise to different molecular weight peaks for the same APP/Aβ biomarkers. They would also appreciate that the exact molecular weight depicted by a peak may vary by up to 10 daltons, including by 1, 2, 3, 4, 5, 6, 7, 8, 9, or 10 daltons, to allow for error. Also a person skilled in the art will recognise that it is possible that salt adducts such as sodium and potassium can form, shifting the predicted molecular weight by 23 and 39 Da respectively.

In yet a further aspect, the present invention provides a software product, the software product comprising: (a) code that accesses data attributed to a sample, the data comprising measurement of at least one biomarker in the sample, the biomarker selected from the group consisting of the biomarkers listed in Table 1 and (b) code that executes a classification algorithm that classifies the AD status of the sample as a function of the measurement. In one embodiment, the classification algorithm classifies AD status of the sample as a function of the measurement of a biomarker selected from the group consisting of 4535, 4631, 9070, or 9980 or combinations thereof (from Table 1).

Other features, objects and advantages of the invention and its preferred embodiments will become apparent from the detailed description, examples and claims that follow.

### BRIEF DESCRIPTION OF FIGURES

FIG. 1a shows representative SELDI-TOF MS spectra extracted from the cellular elements (CE) of blood from an AD subject (top) and an age matched control (HC, bottom). Peaks marked with * are Aβ42 and the corresponding dimer, respectively and these are elevated in AD. Peak 9962/9980 (proposed to be an APP cathepsin D cleavage product), marked † in contrast is elevated in HC. Capture antibody used was WO2.

FIG. 1b shows representative SELDI-TOF MS spectra extracted from the CE of blood from an AD subject (top) and an age matched control (bottom). Peaks due to monomeric (*) and dimeric (**) oxidized Aβ42 are detected by three different antibodies WO2 (epitope 4-8); 4G8 (epitope 17-21); G211 (C-terminus of Aβ42), but not by G210 (C-terminus of Aβ40).

FIG. 1c shows representative SELDI-TOF MS spectra extracted from the CE of blood from an AD subject (top) and a synthetic dimer (bottom) for comparison between the dimer identified in blood and the synthetic dimer.

FIG. 2 shows a map of hierarchical cluster analysis showing two distinct classes of peaks associated with the normal or abnormal processing of APP. At one end of the cluster headed by peak 9962 were species that were higher in HC than in AD. On the other end of the cluster, are peaks due to the Aβ monomer and dimer that were significantly higher in AD. The pattern of the cluster analysis is consistent with two different processing pathways for APP, one an amyloidogenic pathway that is elevated in AD the other a non-amyloidogenic pathway that is elevated in the control subjects.

FIGS. 3a shows box and whiskers plots comparing the intensities of Aβ monomer, dimer and the peak at 9962/9980 Da in HC, MCI and AD. Good separation is shown between the AD and HC groups (Cohen's d: 0.41, 0.76 and 0.73 for the monomer, dimer and 9962/9980 peak, respectively). A PiB SWR threshold of 1.4 was used to separate the groups in PiB-positive (PiB-pos) and PiB-negative (PiB-neg).

FIG. 3b shows box and whiskers plots comparing the respective Aβ monomer and dimer to 9962 Da ratios in HC, MCI, and AD subjects (total of 118 participants). It shows a much better separation between the AD and HC groups is obtained (Cohen's d: 0.76 and 1.03 for monomer and dimer ratios, respectively) than when the peak intensities are examined separately.

FIG. 4 shows the intensities of the peaks assigned to the monomeric oxidized Aβ2 and the corresponding dimer from 118 participants are highly correlated (r=0.79, p<0.0001) HC () ; MCI (+) ; AD (◯).

FIG. 5 shows that Aβ monomer and dimer are highly correlated with clinical, neuropsychometric and biological markers, such as MMSE, memory performance, and brain Aβ burden as measured by PiB-PET, underlying their interrelationship. This reflects the balance in APP processing between the amyloidogenic and non-amyloidogenic pathway that defines AD. HC (); MCI (+); AD (◯).

FIG. 6 shows that Aβ monomer and dimer to 9962 Da ratios are highly correlated with clinical, neuropsychometric and biological markers. It also shows the correlations are better than when either the monomer, dimer, or the 9962 Da peaks are examined separately. HC () ; MCI (+) ; AD (◯)

FIG. 7 shows a representative SELDI-TOF MS spectrum of human CSF, using antibody capture with WO2. The spectrum shows a variety of Aβ species. The intensities of these peaks are decreased in Alzheimer's disease.

### DETAILED DESCRIPTION

A biomarker is an organic biomolecule which is differentially present in a sample taken from a subject of one phenotypic status (e.g., having a disease) as compared with another phenotypic status (e.g., not having the disease). A biomarker is differentially present between different phenotypic statuses if the mean or median expression level of the biomarker in the different groups is calculated to be statistically significant. Common tests for statistical significance include, among others, t-test, ANOVA, Kruskal-Wallis, Wilcoxon, Mann- Whitney and odds ratio. Biomarkers, alone or in combination, provide measures of relative risk that a subject belongs to one phenotypic status or another. Therefore, they are useful as markers for disease (diagnostics), therapeutic effectiveness of a drug (theranostics) and drug toxicity.

The inventors propose that, as Aβ plays a critical role in AD development, it is an appropriate target for a diagnostic for AD. Whilst Aβ biomarkers have been detected previously in CSF and serum, as the inventors propose that Aβ is membrane bound, they consider that Aβ biomarkers detected in cellular (or blood) samples will be far superior to those previously detected in plasma or serum, in terms of sensitivity and specificity.

### Qualification of AD Status

The power of a diagnostic test to correctly predict status is commonly measured as the sensitivity of the assay, the specificity of the assay or the area under a receiver operated characteristic (“ROC”) curve. Sensitivity is the percentage of true positives that are predicted by a test to be positive, while specificity is the percentage of true negatives that are predicted by a test to be negative. An ROC curve provides the sensitivity of a test as a function of specificity. The greater the area under the ROC curve, the more powerful the predictive value of the test. Other useful measures of the utility of a test are positive predictive value and negative predictive value. Positive predictive value is the percentage of actual positives who test as positive. Negative predictive value is the percentage of actual negatives that test as negative.

The biomarkers of the invention can be used in diagnostic tests to assess AD status in a subject, e. g., to diagnose AD disease. The phrase “AD status” includes distinguishing; inter alia, AD v. non-AD and, in particular, AD v. non-AD normal, MCI v. non-AD normal or AD v. MCI. Based on this status, further procedures may be indicated, including additional diagnostic tests or therapeutic procedures or regimens.

The biomarkers of this invention show a statistical difference in different AD statuses of at least p≦0.05, p≦10⁻², p≦10⁻³, p≦10⁻⁴ or p≦10⁻⁵. Diagnostic tests that use these biomarkers alone or in combination show a sensitivity and specificity of at least 50%, at least 55%, at least 60%, at least 65%, at least 70%, at least 75%, at least 80%, at least 85%, at least 90%, at least 95%, at least 98% and about 100%.

### Single Markers

Each Aβ biomarker listed in Table 1 is differentially present in AD versus control and therefore each is individually useful in aiding in the determination of AD status. The method involves, first, measuring the selected biomarker in a subject sample using the methods described herein, e. g. capture on a SELDI biochip followed by detection by mass spectrometry and, second, comparing the measurement with a diagnostic amount or cut-off that distinguishes a positive AD status from a negative AD status. The diagnostic amount represents a measured amount of a biomarker above which or below which a subject is classified as having a particular AD status. For example, if the biomarker is up-regulated compared to normal during AD, then a measured amount above the diagnostic cut-off provides a diagnosis of AD. Alternatively, if the biomarker is down- regulated during AD, then a measured amount below the diagnostic cut-off provides a diagnosis of AD.

As is well understood in the art, by adjusting the particular diagnostic cut-off used in an assay, one can increase sensitivity or specificity of the diagnostic assay depending on the preference of the diagnostician. The particular diagnostic cut-off can be determined, for example, by measuring the amount of the biomarker in a statistically significant number of samples from subjects with the different AD statuses, as was done here, and drawing the cut-off to suit the diagnostician's desired levels of specificity and sensitivity.

### Combinations of Markers

While individual biomarkers are useful diagnostic biomarkers, it has been found that a combination of biomarkers can provide greater predictive value of a particular status than single biomarkers alone. Specifically, the detection of a plurality of biomarkers in a sample can increase the sensitivity and/or specificity of the test. Optimum sensitivity and specificity may be obtained by assaying for a marker of an amyloidogenic pathway and a marker of a non-amyloidogenic pathway. A suitable combination of markers would be the Aβ 1-42 or 1-43 monomer and, or dimer in combination with the APP cathepsin D cleavage product or 9962/9980 molecular weight biomarker identified by SELDI-TOF MS using a WO2 or 4G8 capture antibody.

Detection of Aβ biomarkers

The Aβ biomarkers were discovered using SELDI technology employing ProteinChip arrays from Ciphergen Biosystems, Inc. (Fremont, Calif.) (“Ciphergen”). Briefly, blood samples were collected from subjects diagnosed with AD, MCI and subjects diagnosed as normal, that diagnosis being performed by cognitive testing and by 11C-PIB imaging (the current gold-standard for AD diagnosis). The samples were applied to SELDI biochips (PS10) displaying an Aβ/APP specific antibody (WO2) and spectra of polypeptides in the samples were generated by time-of-flight mass spectrometry on a Ciphergen PBSII mass spectrometer. The spectra thus obtained were analyzed by Ciphergen Express™ Data Manager Software with Biomarker Wizard and Biomarker Pattern Software from Ciphergen Biosystems, Inc. The mass spectra for each group were subjected to scatter plot analysis. A Mann- Whitney test analysis was employed to compare AD and control groups for each protein cluster in the scatter plot, and proteins were selected that differed significantly (p<0.05) between the two groups. This method is described in more detail in the Examples Section.

The biomarkers thus discovered are presented in Table 1.

The biomarkers are characterized by mass-to-charge ratio as determined by mass spectrometry, by the shape of their spectral peak in time-of- flight mass spectrometry and by their binding characteristics to adsorbent surfaces. These characteristics represent inherent characteristics of the biomarkers and not limitations on the process used to discriminate the biomarkers.

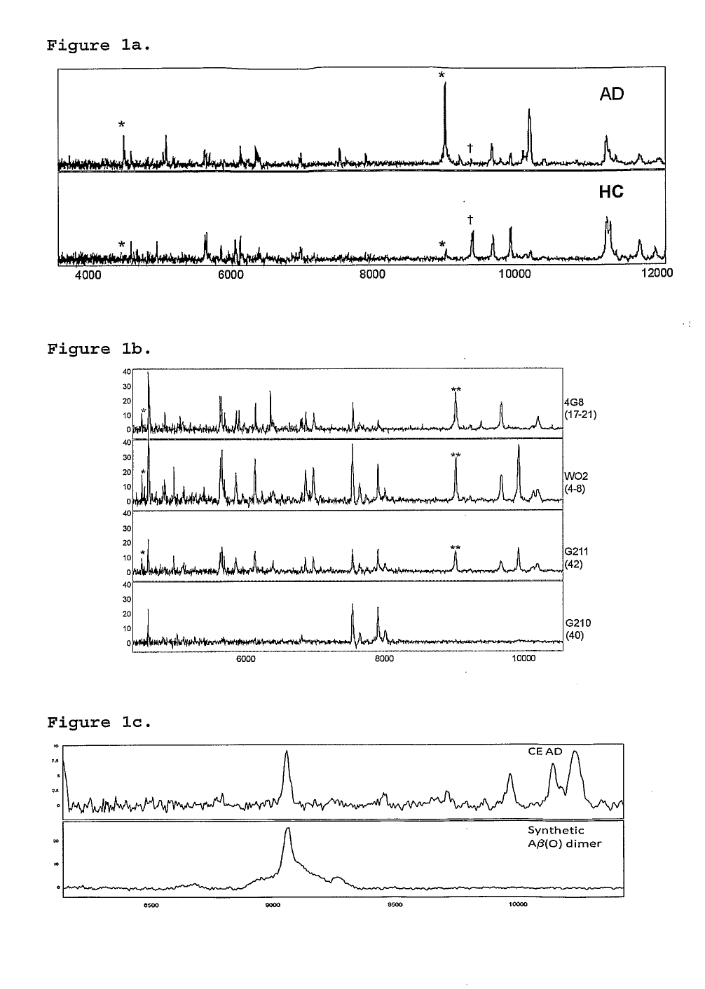

The biomarkers of this invention are further characterized by the shape of their spectral peak in time-of-flight mass spectrometry. Mass spectra showing peaks representing the biomarkers are presented in FIG. 1. The pattern of the peaks differs significantly between samples from subjects diagnosed as AD and non-AD by neuropsychology testing and 11C-PIB analysis. Now the peak pattern for each status has been determined a quick and easy method of diagnosis of AD status is provided, based on checking the pattern of biomarkers presented by a sample against the expected pattern. This pattern recognition may be carried out by eye or using appropriate software, for example Ciphergen's ProteinChip.RTM. software package.

Because the biomarkers of this invention are characterized by mass-to-charge ratio and spectral shape, they can be detected by mass spectrometry without knowing their specific identity. However, if desired, biomarkers whose identity is not determined can be identified by, for example, determining the amino acid sequence of the polypeptides. For example, a biomarker can be peptide-mapped with a number of enzymes, such as trypsin or V8 protease, and the molecular weights of the digestion fragments can be used to search databases for sequences that match the molecular weights of the digestion fragments generated by the various enzymes. Alternatively, protein biomarkers can be sequenced using tandem MS technology. In this method, the protein is isolated by, for example, gel electrophoresis. A band containing the biomarker is cut out and the protein is subject to protease digestion. Individual protein fragments are separated by a first mass spectrometer. The fragment is then subjected to collision-induced cooling, which fragments the peptide and produces a polypeptide ladder. A polypeptide ladder is then analyzed by the second mass spectrometer of the tandem MS. The difference in masses of the members of the polypeptide ladder identifies the amino acids in the sequence. An entire protein can be sequenced this way, or a sequence fragment can be subjected to database mining to find identity candidates.

Once the identity of a biomarker has been determined, the biomarker can be detected by other methods known in the art, e.g., immunoassays or assays for an inherent property of the biomarker, such as an enzymatic activity. The levels of RNA molecules encoding the biomarker may also be measured, e.g., using antisense technology, to determine the extent of biomarker expression.

Proteins frequently exist in a sample in a plurality of different forms. These forms can result from either or both of pre- and post-translational modification. Pre-translational modified forms include allelic variants, splice variants and RNA editing forms. Post-translationally modified forms include forms resulting from proteolytic cleavage (e.g., cleavage of a signal sequence or fragments of a parent protein), glycosylation, phosphorylation, lipidation, oxidation, methylation, cysteinylation, sulphonation and acetylation. The inventors propose that the biomarkers presented in Tables 1 and 2 represent different cleavage products of APP and Aβ and also oligomers and chemically modified forms of these proteins.

In diagnostic assays, the inability to distinguish different forms of a protein has little impact when the forms detected by the particular method used are equally good biomarkers as any particular form. However, when a particular form (or a subset of particular forms) of a protein is a better biomarker than the collection of different forms detected together by a particular method, the power of the assay may suffer. In this case, it is useful to employ an assay method that distinguishes between forms of a protein and that specifically detects and measures a desired form or forms of the protein. Distinguishing different forms of an analyte or specifically detecting a particular form of an analyte is referred to as “resolving” the analyte.

Mass spectrometry is a particularly powerful methodology to resolve different forms of a protein because the different forms typically have different masses that can be resolved by mass spectrometry. Accordingly, if one form of a protein is a superior biomarker for a disease than another form of the biomarker, mass spectrometry may be able to specifically detect and measure the useful form where traditional immunoassay fails to distinguish the forms and fails to specifically detect to useful biomarker.

Whilst a method described in accordance with one embodiment employs SELDI mass spectrometry with immunoassay with an Aβ/APP specific antibody other ways of capturing and assaying Aβ biomolecules will be apparent to those skilled in the art. The Aβ/APP antibody may be any biospecific capture reagent (e.g., an antibody, aptamer or Affibody that recognizes Aβ (in any form). Whilst the invention has been described using Aβ/APP specific monoclonal antibodies WO2 and 4G8 other APP/Aβ specific antibodies could be used. Examples of antibodies or antisera that specifically bind APP or derivative thereof and its analogs, fusions or fragments include monoclonal antibodies 1101.1 (1101.1 was deposited with the American Tissue Type Collection, Rockville, Md. on Apr. 25, 1997 and assigned ATCC No HB12347), 1702.1 (1702.1 was deposited with the American Tissue Type Collection, Rockville, Md. on Jun. 3, 1997 and assigned ATCC No HB12363) and 108.1 (108.1 was deposited with the American Tissue Type Collection, Rockville, Md. on Jun. 3, 1997 and assigned ATCC No HB12362) and antisera BA #1, BA #2, amyloid beta antibodies cat. nos. 0490-1916, 0490-1858, 0490-1857 (ANAWA Biomedical Services & Products, Wangen Switzerland); mouse monoclonal anti-.beta.-amyloid peptide (1-28) (Zymed Laboratories, South San Francisco, Calif.); mouse anti-beta amyloid monoclonal cat no. RDI-BAMYLOID, Research Diagnostics, Inc. Flanders, N.J. Particularly preferred examples of antibodies or antisera that specifically bind .beta.-amyloid precursor protein or derivative thereof and its analogs, fusions or fragments are selected from the following: monoclonal antibodies 1101.1, 1702.1 and 108.1 and antisera BA #1 and BA #2; 6E10, G210, G211, 1ES and other commercially available antibodies. Additionally antibody fragments may also be used.

Preferably, the biospecific capture reagent is bound to a solid phase, such as a bead, a plate, a membrane or an array. After unbound materials are washed away, the captured analytes are detected and/or measured by mass spectrometry. Various forms of mass spectrometry are useful for detecting the protein forms, including laser desorption approaches, such as traditional MALDI or SELDI, and electrospray ionization.

In one embodiment, a sample is analyzed by means of a biochip. Biochips generally comprise solid substrates and have a generally planar surface, to which a capture reagent (also called an adsorbent or affinity reagent) is attached.

Protein biochips are biochips adapted for the capture of polypeptides. Many protein biochips are described in the art. These include, for example, protein biochips produced by Ciphergen Biosystems, Inc. (Fremont, Calif.), Zyomyx (Hayward, Calif.), Invitrogen (Carlsbad, Calif.), Biacore (Uppsala, Sweden) and Procognia (Berkshire, UK).

Protein biochips produced by Ciphergen Biosystems, Inc. comprise surfaces having chromatographic or biospecific adsorbents attached thereto at addressable locations. Ciphergen's ProteinChip.RTM. arrays include NP20 (hydrophilic); H4 and HSO (hydrophobic); SAX-2, Q-10 and LSAX-30 (anion exchange); WCX-2, and CM-10 and LWCX-30 (cation exchange); IMAC-3, IMAC-30 and IMAC-50 (metal chelate); and PS-10, RS100, PS-20 (reactive surface with acyl-imidizole, epoxide) and PG-20 (protein G coupled through acyl-imidizole). Hydrophobic ProteinChip arrays have isopropyl or nonylphenoxy-poly(ethylene glycol) methacrylate functionalities. Anion exchange ProteinChip arrays have quaternary ammonium functionalities. Cation exchange ProteinChip arrays have carboxylate functionalities. Immobilized metal chelate ProteinChip arrays have nitrilotriacetic acid functionalities (IMAC 3 and IMAC 30) or O-methacryloyl-N,N-bis-carboxymethyl tyrosine funtionalities (IMAC 50) that adsorb transition metal ions, such as copper, nickel, zinc, and gallium, by chelation. Preactivated ProteinChip arrays have acyl-imidizole or epoxide functional groups that can react with groups on proteins for covalent binding.

In general, a chip with an adsorbent surface is contacted with the sample for a period of time sufficient to allow the biomarker or biomarkers that may be present in the sample to bind to the adsorbent. After an incubation period, the substrate is washed to remove unbound material. Any suitable washing solutions can be used; preferably, aqueous solutions are employed. The extent to which molecules remain bound can be manipulated by adjusting the stringency of the wash. The elution characteristics of a wash solution can depend, for example, on pH, ionic strength, hydrophobicity, degree of chaotropism, detergent strength, and temperature.

In yet another method, one can capture the biomarkers with a solid-phase bound immuno-adsorbent that has antibodies that bind the biomarkers. After washing the adsorbent to remove unbound material, the biomarkers are eluted from the solid phase and detected by applying to a SELDI biochip that binds the biomarkers and analyzing by SELDI.

“Surface Enhanced Laser Desorption and Ionization” or “SELDI,” as described, for example, in U.S. Pat. No. 5,719,060 and No. 6,225,047, both to Hutchens and Yip. This refers to a method of desorption/ionization gas phase ion spectrometry (e.g., mass spectrometry) in which an analyte (here, one or more of the biomarkers) is captured on the surface of a SELDI mass spectrometry probe.

### Data Analysis

Data generated by desorption and detection of biomarkers can be analyzed with the use of a programmable digital computer. The computer program analyzes the data to indicate the number of biomarkers detected, and optionally the strength of the signal and the determined molecular mass for each biomarker detected. Data analysis can include steps of determining signal strength of a biomarker and removing data deviating from a predetermined statistical distribution. For example, the observed peaks can be normalized, by calculating the height of each peak relative to some reference. The reference can be background noise generated by the instrument and chemicals such as the energy absorbing molecule which is set at zero in the scale.

The computer can transform the resulting data into various formats for display.

Analysis generally involves the identification of peaks in the spectrum that represent signal from an analyte. Peak selection can be done visually, but software is available, as part of that can automate the detection of peaks.

### AD Status

In one embodiment, this invention provides methods for determining the presence or absence of AD in a subject (status: AD v. non-AD). The presence or absence of AD is determined by measuring the relevant biomarker or biomarkers and then either submitting them to a classification algorithm or comparing them with a reference amount and/or pattern of biomarkers that is associated with the particular risk level.

### Determining Risk of Developing Disease

In one embodiment, this invention provides methods for determining the risk of developing disease in a subject. Biomarker amounts or patterns are characteristic of various risk states, e.g., high, medium or low. The risk of developing a disease is determined by measuring the relevant biomarker or biomarkers and then either submitting them to a classification algorithm or comparing them with a reference amount and/or pattern of biomarkers that is associated with the particular risk level.

### Determining Stage of Disease

In one embodiment, this invention provides methods for determining the stage of disease in a subject. Each stage of the disease has a characteristic amount of a biomarker or relative amounts of a set of biomarkers (a pattern). The stage of a disease is determined by measuring the relevant biomarker or biomarkers and then either submitting them to a classification algorithm or comparing them with a reference amount and/or pattern of biomarkers that is associated with the particular stage.

### Determining Course (Progression/Remission) of Disease

In one embodiment, this invention provides methods for determining the course of disease in a subject. Disease course refers to changes in disease status over time, including disease progression (worsening) and disease regression (improvement). Over time, the amounts or relative amounts (e.g., the pattern) of the biomarkers changes. Accordingly, this method involves measuring one or more biomarkers in a subject at two or more different time points, e.g., a first time and a second time, and comparing the change in amounts, if any. The course of disease is determined based on these comparisons.

Similarly, changes in the rate of disease progression (or regression) may be monitored by measuring the amount of a biomarker at different times and calculating the rate of change in biomarker levels. The ability to measure disease state or velocity of disease progression can be important for drug treatment studies where the goal is to slow down or arrest disease progression through therapy.

### Subject Management

In certain embodiments of the methods of qualifying AD status, the methods further comprise managing subject treatment based on the status. Such management includes the actions of the physician or clinician subsequent to determining AD status. For example, if a physician makes a diagnosis of AD, then a certain regime of treatment, such as prescription or administration of a cholinesterase inhibitor might follow. Alternatively, a diagnosis of non-AD or MCI might be followed with further testing to determine a specific disease that the subject might be suffering from. Also, if the diagnostic test gives an inconclusive result on AD status, further tests may be called for.

In another embodiment, this invention provides methods for determining the therapeutic efficacy of a pharmaceutical drug. These methods are useful in performing clinical trials of the drug, as well as monitoring the progress of a subject on the drug. Therapy or clinical trials involve administering the drug in a particular regimen. The regimen may involve a single dose of the drug or multiple doses of the drug over time. The doctor or clinical researcher monitors the effect of the drug on the subject over the course of administration. If the drug has a pharmacological impact on the condition, the amounts or relative amounts (e.g., the pattern or profile) of the biomarkers of this invention changes toward a non-disease profile. Accordingly, this method involves measuring one or more biomarkers in a subject receiving drug therapy, and correlating the amounts of the biomarkers with the disease status of the subject. One embodiment of this method involves determining the levels of the biomarkers at a minimum of two different time points during a course of drug therapy, e.g., a first time and a second time, and comparing the change in amounts of the biomarkers, if any. For example, the biomarkers can be measured before and after drug administration or at two different time points during drug administration. The effect of therapy is determined based on these comparisons. If a treatment is effective, then the biomarkers will trend toward normal, while if treatment is ineffective, the biomarkers will trend toward disease indications.

### Compositions of Matter

In another aspect, this invention provides compositions of matter based on the biomarkers of this invention.

In one embodiment, this invention provides biomarkers of this invention in purified form. Purified biomarkers have utility as antigens to raise antibodies. Purified biomarkers also have utility as standards in assay procedures. As used herein, a “purified biomarker” is a biomarker that has been isolated from other proteins and peptides, and/or other material from the biological sample in which the biomarker is found. Biomarkers may be purified using any method known in the art, including, but not limited to, mechanical separation (e.g., centrifugation), ammonium sulphate precipitation, dialysis (including size-exclusion dialysis), size-exclusion chromatography, affinity chromatography, anion-exchange chromatography, cation-exchange chromatography, and metal-chelate chromatography. Such methods may be performed at any appropriate scale, for example, in a chromatography column, or on a biochip.

### Use of Biomarkers for AD in Screening Assays

The biomarkers can be used to screen for compounds that modulate the expression of the biomarkers in vitro or in vivo, which compounds in turn may be useful in treating or preventing AD in subjects. In another example, the biomarkers can be used to monitor the response to treatments for AD. In yet another example, the biomarkers can be used in heredity studies to determine if the subject is at risk for developing AD.

For the purposes of this specification it will be clearly understood that the word “comprising” means “including but not limited to”, and that the word “comprises” has a corresponding meaning.

It will be clearly understood that, although a number of prior art publications are referred to herein, this reference does not constitute an admission that any of these documents forms part of the common general knowledge in the art, in Australia or in any other country.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

Embodiments of the present invention will now be described in the following non-limited examples.

### Examples

### Example 1

### Participants

Fifty-two elderly individuals with well documented normal cognitive function, 43 patients with mild to moderate AD, and 23 subjects with MCI were recruited for the study (Table 2). All AD patients met NINCDS-ADRDA criteria for probable AD. MCI subjects met the Petersen criteria of subjective and objective cognitive difficulties, predominantly affecting memory, in the absence of dementia or significant functional loss. All patients were recruited from the Austin Health Memory Disorders and Neurobehavioural Clinics or the Healthy Aging Study (Mental Health Research Institute). None of the participants were receiving or had ever received anti-Aβ medication.

Written informed consent was obtained prior to participation. Approval for the study was obtained from the Austin Health Human Research Ethics Committee.

TABLE 2
Participants (means (S.D.) for groups by classification)

| | HC | MCI | AD |
|---|---|---|---|
| | (n = 52) | (n = 23) | (n = 43) |
| Age | 73.2 (7.7) | 70.9 (9.6) | 72.7 (10.7) |
| MMSE | 29.0 (1.1) | 26.3 (2.4)* | 21.8 (4.1)* |
| Male/Female | 25/27 | 9/14 | 20/23 |
| % ApoE ε4 | 29% | 48% | 73% |
| Neocortical PiB | 1.4 (0.4) | | 2.3 (0.4)* |
| SUVR | | 1.9 (0.6)* | |
| Composite | −0.1 (0.9) | | −3.4 (0.6)* |
| Memory | | −2.4 (1.0)* | |

Significantly different from controls (p<0.05).

HC represents healthy control.

### Blood Sample Preparation

Whole blood was collected by phlebotomy in EDTA vacutainers (6 mL K2EDTA, Greiner Bio-One, Australia) and processed within 30 min of procurement. Vacutainers were spun at 3500 rpm at 4° C. for 30 min, The upper layer of plasma was then removed, and both cellular and plasma fractions were kept at −80° C. until used.

An aliquot of each fraction (10 μL) was mixed with 10 μL of urea 8 M, and 120 μL of 0.5% TritonX-100/PBS, and placed for 15 min in an ultra-sound bath with ice. The purpose of adding a denaturant and detergent was to break up any potential protein/protein or protein/membrane interactions involving any APP/Aβ fragments. A variety of denaturants and detergents were assessed over a large concentration range to identify the conditions that reproducibly gave the best signal to noise in the mass spectra. The extracted material was then analysed in triplicate by SELDI-TOF MS, the operator was blinded to the disease status of the subjects, using antibody capture (WO2 epitope Aβ residues 4-8 and 4G8 epitope Aβ residues 17-21). These are generic anti-Aβ antibodies able to capture most Aβ species.

PS10 ProteinChip arrays were used for the SELDI-TOF Specific antibodies (2 μl of either 4G8 or WO2) were added to the arrays in PBS (0.25 mg/mL). To confirm that the binding observed was not due to non-specific binding control spectra using a non-specific IgG antibody were also obtained. Chips were then incubated overnight at 4° C. in a humidity chamber.

Antibodies were then removed and blocking buffer (0.5 M ethanolamine in PBS) was added (5 μL) and arrays were incubated for 30 min. After the removal of the blocking buffer, each array was washed for 5 min with 50 μL of 0.5% Triton X-100/PBS (wash-buffer). The solvent was then removed, and the arrays were washed for 5 min with 50 μL of PBS. All samples were analysed in triplicate. Samples (130 μL) were added to each spot and the arrays were incubated at room temperature for 3 hours. The samples were then removed, and each spot was washed twice with 100 μL of wash-buffer for 10 sec, followed by a wash with 100 μL PBS twice for 10 sec as well. Finally, the arrays were washed twice with 100 μL HEPES 1 mM for 10 sec. The array was then air-dried. One μL of sinapinic acid (SPA, 50% saturated in 50% (v/v) acetonitrile and 0.5% in TFA) was applied to each spot twice. The array was air-dried between each application. All incubations and washes were performed on a shaking table

Chips were analyzed in a PBSIIC, SELDI-TOF MS, and peaks were analysed using Ciphergen ProteinChip software 3.1. The distributions of the peak intensities in the spectra showed skewness to either left or right. By taking the logarithm of the peak intensities, skewness was substantially reduced, and the distributions met criteria for normality.

Preparation of Aβ₁₋₄₂Met₃₅(O) dimer

Resin-bound Aβ₁₁₋₄₂Met₃₅(O) was prepared according to standard methods (Tickler et al., (2001) J Pept Sci 7:488-494; Barnham et al., (2003) J Biol Chem 278:42959-42965. 2003). Dityrosine was prepared according to the previously reported method (Skaff et al., (2005) J Org Chem 70:7353-7363). Fmoc protection of dityrosine and incorporation into SPPS of the Aβ₁₋₄₂Met₃₅(O) dimer was performed according to the previously reported method (Kok et al., (2009) Chem Commun, DOI: 10.1039/b912784d).

### Genotyping

ApoE genotype was determined by PCR amplification of genomic DNA.

### Neuropsychological Assessments

All subjects evaluated undertook a variety of neuropsychological tasks, designed to assess a broad range of cognitive domains commonly affected by AD and aging, such as global cognition and memory. In addition to the Mini Mental State Examination (MMSE) and CDR tests, memory was tested using delayed recall of the California Verbal Learning Test-Second edition (CVLT-II) and the Rey Complex Figure Test (RCFT).

A composite episodic memory score was generated by taking the average of the Z scores (generated using 50 healthy older peoples with negative PIB scans as the reference) for the memory tasks.

All subjects undertook a variety of neuropsychological tasks, designed to assess a broad range of cognitive domains commonly affected by AD and aging. In addition to the Mini-Mental State Examination (MMSE) and CDR tests, memory was tested using delayed recall of the California Verbal Learning Test-Second edition (CVLT-II), and the Rey Complex Figure Test (RCFT), while frontal function was tested using FAS (letter fluency), Category fluency (animals+boys), verbal fluency switching task (Fruit furniture switching), and stroop incongruent. Using the results of 65 healthy older people with negative PiB scans as the reference, a composite episodic memory score was generated by taking the average of the Z scores for the memory tasks, 26 and an executive function score was generated by taking the average of the Z scores for the frontal function tasks.

### Neuroimaging

All subjects underwent a 3D spoiled gradient echo (SPGR) T1-weighed MRI acquisition for screening and subsequent co-registration with the PET images.

Production of ¹¹C-PIB and PET scans were performed at the Centre for PET, Austin Hospital, as previously described. Briefly, a 30-minute acquisition emission PET scan was acquired starting at 40 minutes after the administration of 370 MBq of ¹¹C-PiB. Regional Standardized uptake value (SUV), defined as the decay-corrected brain radioactivity concentration, corrected for injected dose and body weight, was normalized to the cerebellar cortex to obtain SUV Ratios (SUVR). Neocortical Aβ burden was expressed as the average SWR of the area-weighted mean for the following cortical ROIs: frontal (consisting of dorsolateral prefrontal, ventrolateral prefrontal, and orbitofrontal regions), superior parietal, lateral temporal, lateral occipital, and anterior and posterior cingulated.

All subjects underwent a 3D spoiled gradient echo (SPGR) T1-weighed MRI acquisition for screening and subsequent co-registration with the PET images. As described elsewhere, T1W MR images for each subject were classified into GM, WM and CSF using an implementation of the expectation maximization segmentation algorithm. The MNI single-subject MRI brain template and corresponding AAL template and tissues priors were spatially normalized to each participant to automatically obtain a parcellation for each selected atlas into GM, WM and CSF. The measured grey matter volumes were normalized for head size using the total intracranial volume, defined as the sum of GM, WM and CSF volumes. The volume results are presented as the proportion of total intracranial volume.

Statistical analysis

Continuous variables for the groups were tested for normality of distribution using the Shapiro-Wilk test and visual inspection of variable histograms. Statistical evaluations were performed using a Dunnet's test to compare each group with controls, and a Tukey-Kramer HSD test to establish differences between group means. Pearson product-moment correlation analyses were conducted between the different variables. In order to investigate the patterns and inter-relationships between the series of spectrum peaks, a hierarchical cluster analysis using an average linkage method was performed. Data are presented as mean ± standard deviation (SD) unless otherwise stated.

Detection of Aβ biomarkers in CSF

PS20 ProteinChip array were used where a volume of 2 μl of antibody (WO2) in PBS (0.25 mg/mL) was added to the spots of the PS20 chip and it was incubated in the humidity chamber at 4° C. overnight. The antibody was removed and blocking buffer (0.5 M ethanolamine in PBS) was added (5 μL), the array was incubated for 30 min. The blocking buffer was removed and each spot was washed with 0.5% Triton X-100/PBS (wash buffer) for 5 min. The solvent was removed and the spots were washed with PBS for 5 min. A volume of 12 μl of neat CSF sample was added to each spot, the array was incubated at room temperature for 3 hours. The samples were removed and each spot was washed with wash-buffer followed by a PBS wash and a HEPES wash also, each wash was performed twice.

A volume of 1 μL of sinapinic acid (SPA, 50% saturated in 30% (v/v) acetonitrile 10% IPA (isopropyl alcohol) and 0.5% in TFA) was applied to each spot twice. The array was air-dried between each application.

All the incubations and washes were performed on a shaking table.

### Results

Demographics, clinical, neuropsychological and neuroimaging characteristics of the participants are reported in Table 2. There were significant differences between the aged matched control group (HC) and AD and MCI in MMSE and memory scores. The AD group had the higher prevalence (>70%) of ApoE e4 allele carriers. There was no significant difference in age or in gender distribution between groups.

Blood was collected from all participants and fractionated into plasma and CEs; fractions were then treated with an aqueous solution of urea and Triton-X 100. The purpose of adding urea and detergent was to break up any potential protein/protein or protein/membrane interactions involving any APP/Aβ fragments. The material was then analysed on a SELDI-TOF MS using antibody capture (WO2 epitope Aβ residues 4-8 and 4G8 epitope Aβ residues 17-21). SELDI-TOF MS of the plasma fraction failed to resolve any peaks of significance.

The spectra of the CE material from both diseased and healthy subjects contained a large number of peaks ranging in size from 3.5-16 KDa, consistent with a variety of APP/Aβ fragments being present in the CEs. Moreover, the spectra obtained from AD patient blood was significantly different to that of HC (FIG. 1a). The m/z of the peaks with intensities that were found to be significantly different between the HC and AD groups are listed in Table 2. SELDI-TOF MS of the plasma fraction did not resolve any peaks that were significantly different between the control and disease groups; nor were any peaks due to species normally associated with AD (i.e. Aβ) observed (not shown).

To confirm that the peaks at 4529/4535 and 9058/9070 corresponded to Aβ1-42 species, SELDI-TOF spectra were collected using the G210 (an Aβ40 specific antibody) and G211 (an Aβ42 species antibody). As shown in FIG. 1b, the peaks that were assigned to Aβ42 and the corresponding dimer are detected by three different Aβ antibodies (4G8, WO2, G211) but are not detected by the Aβ40 specific antibody G210. The molecular weights of the peaks with intensities that were found to be significantly different between the HC and AD groups are listed in Table 1.

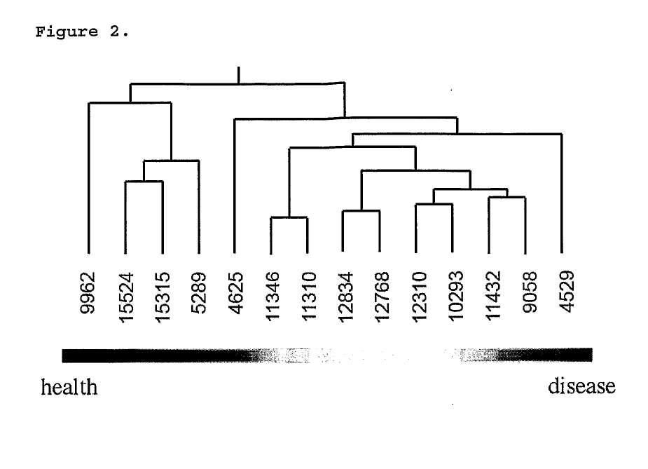

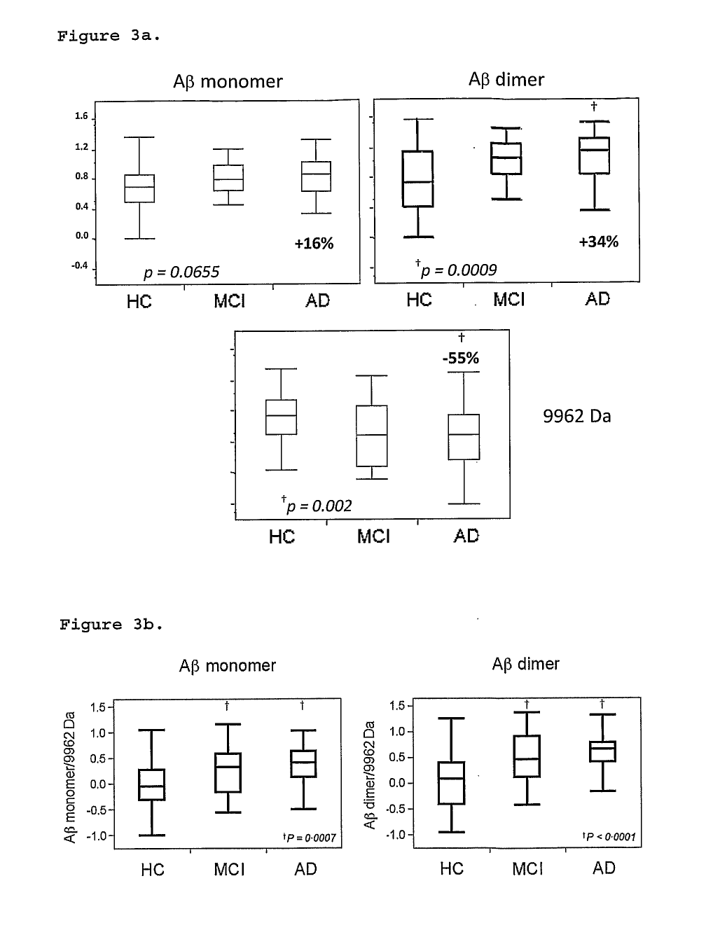

Hierarchical clustering of the CE WO2 data set of AD and AC revealed two distinct groups, (FIG. 2) with one group elevated in disease while the other is elevated in the healthy cohort. At one end of the cluster analysis are a number of peaks that are elevated in AD whose molecular weights are consistent with species prominent in the amyloidogenic pathway commonly associated with AD, including Aβ1-42 and 1-43 (Molecular weight 4529/4535 and 4625/4631 Da respectively). The intensities of the peaks due to Aβ1-42 are significantly increased by 16% in the AD subjects as compared to the controls (FIG. 3a). Additionally the peak at 9058/9070 Da corresponds to the molecular weight of the dimer of Aβ1-42 is also elevated by 34% in AD in a highly statistical manner (FIG. 3a).

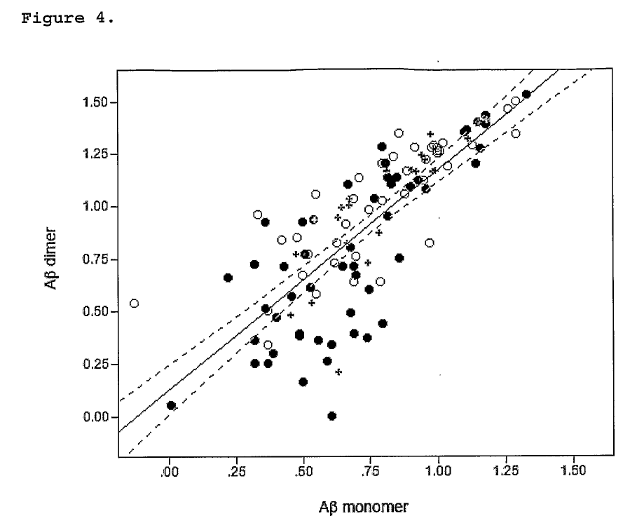

As would be expected there is a strong statistically significant correlation between the amount of monomeric Aβ1-42 detected in blood and the corresponding dimer (r=0.78, p<0.0001) (see FIG. 4).

To confirm that the peaks at m/z 4529 and 9058 corresponded to Aβ42 species, SELDI-TOF spectra were collected using the antibodies G210 (Aβ40 specific) (Ida et al., (1996) J Biol Chem 271:22908-22914) and G211 (Aβ42 specific) (Ida at al., supra). As shown in FIG. 1b, the peaks that were assigned to Aβ42 and the corresponding dimer were detected by three different Aβ antibodies (4G8, WO2, G211) but were not detected by the Aβ40 specific antibody G210. To further characterize the Aβ dimer an oxidized Aβ dimer was synthesised (the sulfur atom of Met35 is oxidized to a sulfoxide) where the two Aβ peptide chains are covalently cross-linked with a dityrosine moiety at residue number ten. As can be seen from FIG. 1c, the SELDI MS of the molecular weight of this synthetic dimer is the same as the dimer elevated in AD blood.

At the opposite end of the cluster is the peak at 9962/9980 Da which is significantly elevated (55% higher) in AC compared to control. This molecular weight corresponds to an APP cathepsin D cleavage product. Cathepsin D is reduced in AD patients, reinforcing the position that the cathespin D cleavage product would be decreased in AD patients, as has been found here. Therefore the observed distribution in the cluster analysis is consistent with the notion that there are differential processing pathways for APP between diseased and healthy subjects.

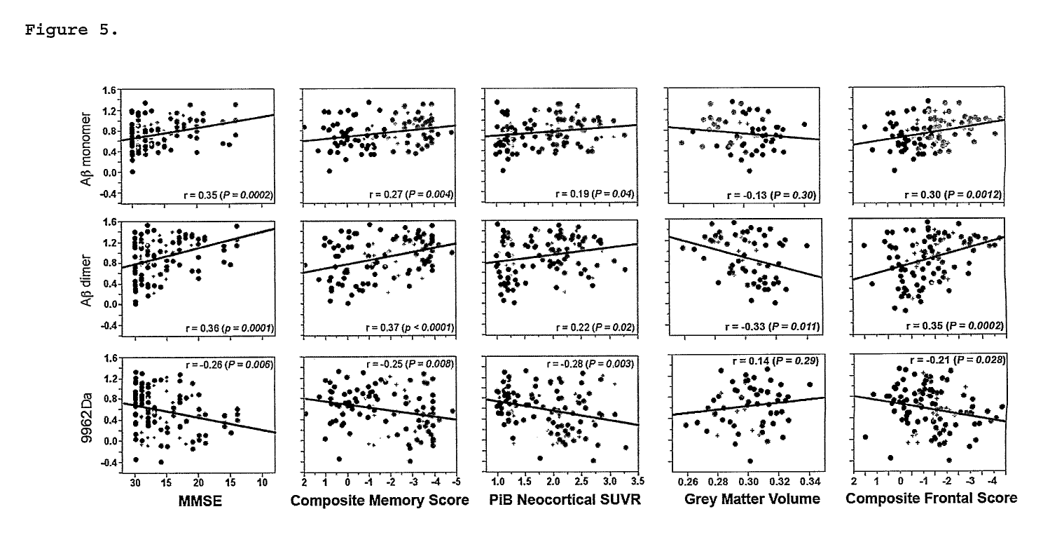

Not only are the Aβ monomer and dimer elevated in AD blood as compared to control but there are highly statistically significant correlations between the levels of these species in the blood and other clinical, neuropsychometric and biological markers used to diagnose AD. Those include Mini-Mental State examination MMSE (r=−0.35 and −0.36; p=0.0002 and 0.0001, formonomeric and dimeric Aβ, respectively), memory impairment (r=−0.27 and −0.37, p=0.004 and 0.0001, for monomer and dimer, respectively (FIG. 5).

Furthermore, when all the subjects including the MCI group are separated into PiB-positive and PiB-negative groups using a Neocortical SUVR threshold of 1.4, obtained by unbiased statistical approaches such as hierarchical cluster analysis or partition models for the determination of a Neocortical PiB ‘cut-off’ level applied to the HC group, the monomer (P=0.013) and dimer (P=0.0002) are both significantly elevated within the PiB-positive group when compared to the negative group.

There are also significant correlations between m/z 9962 Da and the clinical neuropsychometric and biological markers (respectively r=−0.26 (P=0.006); −0.25 (P=0.01); −0.28 (0.003) for MMSE, memory impairment and brain Aβ burden) (FIG. 5). This peak was significantly (P=0.005) elevated in the PiB-negative group (FIG. 5). As this peak is higher in controls relative to diseased subjects, the correlations are of opposite sign to those of monomeric and dimeric Aβ.

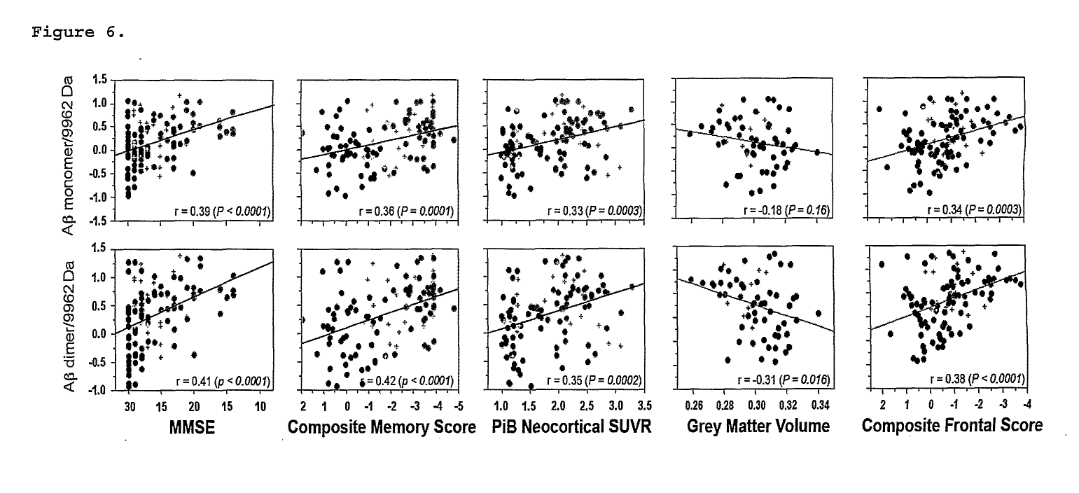

As the 9962 m/z peak and the Aβ dimer peak reflect a balance between two different processing pathways, we further examined the ability of the ratio of the monomer and dimer to the 9962 Da peak to discriminate between AD and controls. The distinction between AD and controls for the monomer and dimer ratios was better than when using the peaks intensities independently (P=0.0007 and <0.0001, for monomer and dimer ratios respectively) (FIG. 6), and the correlations with clinical markers of AD were also improved: MMSE (r=−0.39 and −0.41, P=<0.0001, for monomer and dimer ratios, respectively), memory impairment (r=−0.36 and −0.42, P=<0.0001, for monomer and dimer ratios, respectively), and brain Aβ burden as measured by ¹¹C-PiB PET (r=0.33 and 0.35, P=0.0003 and 0.0002, for monomer and dimer ratios, respectively) (FIG. 6). This improvement was also reflected in a larger effect size for the ratio compared to the one obtained with the Aβ dimer alone (1.03 and 0.76, respectively).

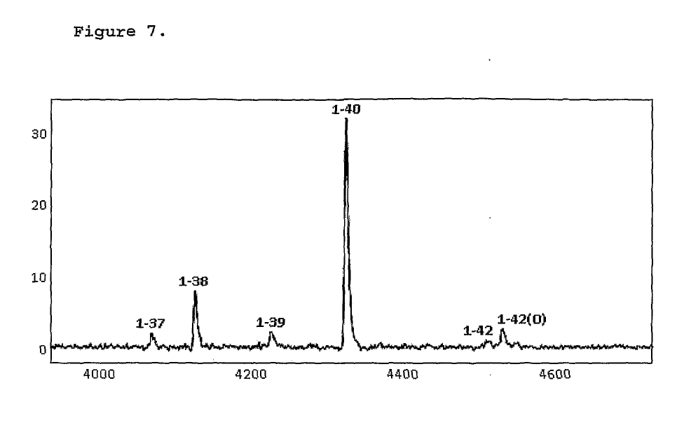

FIG. 7 shows that Aβ biomarkers are detectable in CSF. The peaks decrease in intensity as Alzheimer's disease progresses. Decreases in CSF Aβ levels, as detected by ELISA methods, is one of the current “gold standard” biomarkers of AD. Similar effects are observed using the present methods, thereby adding to the validity of the present methods.

### Discussion

Recent studies have indicated that synaptotoxic Aβ dimers are elevated in AD brains, yet none of the Aβ biomarker protocols currently used clinically, e.g. PIE PET imaging or Aβ ELISAs in CSF are able to detect such species. Using SELDI-TOF MS technology it is possible to detect a dimeric form of Aβ in human blood and show that the levels of the dimer are significantly elevated in AD (FIG. 1) and correlate with clinical markers of the disease (FIG. 5).

Conversely, a peak at 9962/9980 Da is lower in diseased subjects (FIG. 1a) and is inversely correlated with clinical markers of AD (FIG. 5). This molecular weight does not correspond to any obvious APP fragment—it is too small to be a fragment resulting from β-secretase activity, and too big to be the result of γ-secretase activity—nor does the mass correspond to any Aβ-like aggregate. This suggests that the fragment is generated via an alternative, non-amyloidogenic processing pathway. Definitive identification of this fragment will require isolation and amino acid sequencing, but the cluster analysis (FIG. 2) does give some clues to the potential identification of this fragment. It has previously been reported that the activity of cathepsin D is decreased in the blood of AD subjects. Cathepsin D is an aspartyl protease that cleaves APP at a number of different sites including at Ser627, Phe765, Glu766 and Met768. Cleavage at these sites would give rise to a number of 15 KDa fragments; subsequent ε-cleavage of these fragments by γ-secretase at Met722 would give rise to a 10 KDa fragment. There is also a cathepsin D cleavage site at Va1669, subsequent cleavage by γ-secretase at the epsilon site would give rise to a 5 KDa fragment.

As can be seen from Table 1 and the cluster analysis (FIG. 2) a number of related fragments with similar masses are detected as being elevated in the blood of the control subjects. The spectrum of APP fragments observed by SELDI-TOF MS in the cluster analysis is consistent with there being two distinct processing pathways for APP, an amyloidogenic pathway which predominates in AD and a non-amyloidogenic pathway which predominates in healthy subjects (FIG. 2). Both these pathways occur physiologically and it could be argued that the progression to AD is the result of a shift in the processing of APP from the non-amyloidogenic to the amyloidogenic pathway. The genetics of early onset AD support this model. The recent publication that β-secretase activity in platelets is increased in AD as compared to controls is consistent with the amyloidogenic pathway being “favored” in AD.

Given that the onset of the disease may predate clinical symptoms of AD by many years, the lack of valid biomarkers has hampered the development of effective therapies for AD. A number of potential AD therapeutic strategies targeting Aβ and its oligomers (so called disease-modifying drugs) are currently being investigated including immunotherapy designed to promote Aβ clearance, secretase inhibitors which prevent Aβ generation, scyllo-inositol which is reported to inhibit toxic Aβ oligomers binding to membranes and PBT2 a second generation MPAC that inhibits the formation of toxic Aβ oligomers. The assessment of outcomes of the clinical trials is often difficult to define as they rely on highly variable neuropsychometric tests. To overcome the variability that is inherent in these tests, large sample sizes and long timeframes are required to observe subtle changes in subjects' performance, dramatically increasing the cost of these trials. The ability to detect preclinical or early stage disease through reliable laboratory and neuroimaging biomarkers for AD would enable more efficient clinical trials to be designed and monitored. Ideally a biomarker should reflect a disease specific process and be detected in an easily collected tissue.

The processing of APP to generate Aβ and its subsequent aggregation to form toxic oligomers are now seen as fundamental to the development of AD. We have previously demonstrated that Aβ oligomers have a high affinity for lipid membranes and the ability of these oligomers to bind membranes correlates with their toxicity. Therefore we hypothesized that the most likely tissue to contain Aβ oligomers would be one rich in lipid membranes. The most easily accessed tissue is blood and the fractionation procedures we employed were deliberately kept simple to reflect the standard protocol used in clinical laboratories worldwide to perform plasma-based assays. However in this instance we analysed the usually discarded membrane-rich CE fraction. The data presented here establishes that disease relevant APP/Aβ based biomarkers are likely to be found in the membrane fraction of blood. Given that the blood borne biomarkers correlate with disease progression they hold the promise of providing a simple yet effective way of monitoring the success or otherwise of the various disease modifying therapies targeting Aβ/APP processing.

Because the molecular changes occur well before the phenotypical manifestation of disease, identification of specific biomarkers for particular traits of the pathological process will permit early intervention with disease-modifying medications. Further characterization of the different species in AD and controls is warranted, while ongoing longitudinal studies will help elucidate how this markers change over time and how do they relate to cognitive decline.

1. A biomarker for qualifying Alzheimer's disease status, said biomarker being detectable in a biological sample containing blood cellular elements and being derived from amyloid precursor protein or amyloid β peptide.

2. A biomarker according to claim 1 comprising an Aβ1-42 or Aβ1-43 species (monomer) and, or an Aβ1-42 dimer, wherein an increase in the monomer or dimer compared to control is predictive of AD.

3. A biomarker according to claim 1 comprising an APP cathepsin D cleavage product or comprising biomarker identifiable by a peak of molecular weight of about 9962 or 9980 Daltons when identified by SELDI-TOF MS utilising antibody WO2, wherein a decrease in the APP cathepsin D cleavage product or 9962 or 9980 biomarker is predictive of AD status.

4. A biomarker for qualifying Alzheimer's disease status, said biomarker being detectable in a biological sample containing blood cellular elements and being selected from the list of biomarkers presented in Table 1:
TABLE 1
The molecular weight of peaks identified utilizing the antibodies
WO2 and 4G8 in the SELDI-TOF MS where the intensities were
statistically different between AD and AC subjects:

| | |
|---|---|
| WO2 | 4G8 |
| Peak | | Peak | |
|---|---|---|---|
| molecular | | molecular | |
| weight | p value | weight | p value |
| 4529/4535 | 0.07/0.0008 | | |
| 4625/4631 | 0.003/0.0107 | | |
| 5289/5297 | 0.028/0.0439 | | |
| 9058/9070 | <0.0001/0.0005 | 9058/9070 | 0.001/0.0011 |
| 9962/9980 | 0.002/0.0021 | | |
| 10255/10293 | 0.0037/0.004 | 10254/10292 | 0.022/0.034 |
| 11310/11330 | 0.001/0.0014 | 11310/11330 | 0.044/0.0435 |
| 11346/11364 | 0.001/0.0055 | | |
| 11432/11453 | 0.014/0.0163 | | |
| 12310/12330 | 0.002/0.0082 | 12310/12330 | 0.024/0.0241 |
| 12768/12787 | 0.003/0.0028 | | |
| 12834/12859 | 0.008/0.0114 | | |
| 15315/15339 | 0.07/0.0135 | | |
| 15524/15546 | 0.06/0.0057. | | |

5. A method for qualifying Alzheimer's disease status in a subject, the method comprising assaying a biological sample from the subject, the biological sample comprising blood cellular elements, for a biomarker according to claim 1 and correlating the result of the assay with Alzheimer's disease status.

6. (canceled)

7. A method for qualifying Alzheimer's disease status in a subject, the method comprising assaying a biological sample from the subject, the biological sample comprising blood cellular elements comprising assaying for a plurality of biomarkers according to claim 1 and correlating the result of the assay with Alzheimer's disease status.

8. A method according to claim 7 comprising assaying a first biomarker involved in an amyloidogenic pathway and a second biomarker involved in a non-amyloidogenic pathway, wherein an increase in the first biomarker and a decrease in the second biomarker compared to control is predictive of Alzheimer's disease status.

9. A method according to claim 7 comprising assaying for all biomarkers in a sample and comparing the pattern of biomarkers with patterns

10. (canceled)

11. A biomarker, according to claim 1, wherein the biological sample containing blood cellular elements comprises red blood cells, white blood cells, platelets or combinations thereof.

12. A biomarker, use according to claim 11, wherein the biological sample is a serum sample.

13. A biomarker, according to claim 11, wherein the sample is treated with urea and non-ionic detergent before assaying for biomarkers.

14. A method according to claim 5 wherein the AD status is selected from AD, non-dementia, non-AD dementia and MCI, non-AD dementia, Lewy body dementia (LBD) and frontotemporal dementia (FTD).

15. The method according to claim 5 for managing subject treatment based on AD status.

16. The method according to claim 5 for determining efficacy of drugs in treating AD.

17. A kit comprising a solid support comprising at least one capture agent attached thereto, wherein the capture reagent binds at least one biomarker according to claim 1 and instructions for using the solid support to detect the at least one biomarker.

18. A kit according to claim 17, wherein the solid support comprising a capture agent is a SELDI chip.

19. A kit according to claim 17, wherein the capture agent is a Aβ/APP specific antibody.

20. A kit according to claim 19, wherein the Aβ/APP specific antibody is WO2 or 4G8.

21. A method according to claim 5, wherein the biomarker is assayed by capturing the biomarker on an adsorbant surface of a SELDI probe and detecting the captured biomarkers by laser desorption-ionization mass spectrometry.

22. A method according to claim 21, wherein the adsorbant is an antibody specific for APP and Aβ.

23. (canceled)

24. A method for qualifying Alzheimer's disease status in a subject, the method comprising assaying a biological sample from the subject, the biological sample comprising blood cellular elements comprising assaying for a plurality of biomarkers according to claim 4 and correlating the result of the assay with Alzheimer's disease status.
